(19) United States
(12) Patent Application Publication
(10) Pub. No.: US 2025/0255753 A1
(43) Pub. Date: Aug. 14, 2025

(54) DEVICE AND METHOD FOR POST-OPERATIVE EAR CANAL TREATMENT

(71) Applicant: BIOMEDCRAFT DESIGNS, INC., Gerald, MO (US)

(72) Inventors: Dale Rice, Pacific Palisades, CA (US); Courtney Christine Joan Voelker, Los Angeles, CA (US)

(73) Assignee: BIOMEDCRAFT DESIGNS, INC., Gerald, MO (US)

(21) Appl. No.: 18/726,224

(22) PCT Filed: Jan. 4, 2022
(86) PCT No.: PCT/US22/11132

§ 371 Date: Jul. 2, 2024

Related U.S. Application Data
(60) Provisional application No. 63/177,089, filed on Apr. 20, 2021.
(60) Provisional application No. 63/133,757, filed on Jan. 4, 2021.

(51) Int. Cl. A61F 11/20; A61M 31/00

(52) U.S. Cl. CPC A61F 11/20; A61M 31/007; A61F 2210/0004; A61F 2250/0067; A61M 2210/0662

(57) ABSTRACT

A device (100) for post-operative ear canal treatment, comprising a plurality of sides (102, 104, 106, 108, 110), each having a length of less than 5 millimeters, the plurality of sides coupled to an adjacent side at a first end and to another adjacent side at a second end, the plurality of side forming a compressible tubular, the plurality of sides each formed from a material that dissolves over time when exposed to an ear canal and which releases a predetermined ear medication as it dissolves and a delivery system configured to controllably install the device in the ear canal with a restriction on the extent to which the delivery system can be inserted into the ear canal, so as to prevent injury to an ear drum.

### RELATED APPLICATIONS

The present application claims priority to and benefit of U.S. Provisional Patent applications 63/133,757, filed Jan. 4, 2021, and 63/177,089, filed Apr. 20, 2021, each of which are hereby incorporated by references for all purposes as if set forth herein in their entireties.

### TECHNICAL FIELD

The present disclosure relates generally to ear canal treatment, and more specifically to devices and methods for post-operative ear canal treatment.

### BACKGROUND OF THE INVENTION

Treating the ear canal is difficult because it can become infected or otherwise develop complications. For example, stenosis and scarring can result from infection or can occur during post-operative healing.

### SUMMARY OF THE INVENTION

A device for post-operative ear canal treatment is disclosed that includes a plurality of sides, each having a length of less than 5 millimeters, the plurality of sides coupled to an adjacent side at a first end and to another adjacent side at a second end, where the plurality of sides forms a compressible tubular. The plurality of sides are each formed from a material that dissolves over time when exposed to an ear canal and which releases a predetermined ear medication as it dissolves. A delivery system is configured to controllably install the device in the ear canal with a restriction on the extent to which the delivery system can be inserted into the ear canal, so as to prevent injury to an ear drum.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE SEVERAL VIEWS OF THE DRAWINGS

Aspects of the disclosure can be better understood with reference to the following drawings. The components in the drawings may be to scale, but emphasis is placed upon clearly illustrating the principles of the present disclosure;

FIG. 1 is a side view of a surgical implant, in accordance with an example embodiment of the present disclosure;

FIG. 2 is a bottom view of a surgical implant, in accordance with an example embodiment of the present disclosure;

FIG. 3 is a side view of a deployed surgical implant, in accordance with an example embodiment of the present disclosure;

FIG. 4 is a side view of a delivery system, in accordance with an example embodiment of the present disclosure;

FIG. 5 is a diagram of a surgical implant delivery system in an ear canal with a surgical implant, in accordance with an example embodiment of the present disclosure; and

FIG. 6 is a diagram of an method for utilizing a surgical implant delivery system in an ear canal with a surgical implant, in accordance with an example embodiment of the present disclosure.

### DETAILED DESCRIPTION OF THE INVENTION

In the description that follows, like parts are marked throughout the specification and drawings with the same reference numerals. The drawing figures may be to scale and certain components can be shown in generalized or schematic form and identified by commercial designations in the interest of clarity and conciseness.

Propel® is a post-surgical implant device manufactured by Intersect ENT of Menlo Park, CA. It is configured to for use in the ethmoid sinus, usually at the conclusion of ethmoid sinus surgery, and is used to maintain the space between the middle turbinate and the lamina papyracea (the ethmoid sinus). Propel eludes mometasone (a steroid) into the tissues for a period of time to aid healing. After about 2-3 weeks it slowly disintegrates and is washed away.

The present disclosure is directed to a post-surgical implant for the ear canal, which can be similar to the design and materials used for the Propel device, or which can be modified as needed to improve the applicability of the post-surgical implant for otological applications. In one example embodiment, it can be placed into the external auditory canal (ear canal) at the conclusion of ear surgery, such as where the ear canal skin has been manipulated or at other suitable locations. In this manner, it can be used to hold the ear canal open while it heals. The post-surgical implant can be fabricated from a bio-absorbable copolymer, such as poly (L-lactide-co-glycolide), polyhydroxyalkanoates (PHAs) such as poly-3-hydroxybutyrate (PHB or PH3B), polyhydroxyvalerate (PHV), and polyhydroxyhexanoate (PHH), poly(3-hydroxybutyrate-co-3-hydroxyvalerate, polyglycolic acid (PGA), poly-l-lactic acid (PLLA), poly-d-lactic acid, polycyanoacrylates, polyanhydrides, polypropylene fumarate or other suitable materials. Biodegradation can be accomplished by synthesizing polymers with hydrolytically unstable linkages in the backbone. This is commonly achieved by the use of chemical functional groups such as esters, anhydrides, orthoesters and amides, by synthesizing the polymer by ring opening polymerization or in other suitable manners.

The traditional way to treat the ear canal after surgery is to fill the ear with Gelfoam® from Pfizer, which stays in place several weeks and which completely occludes the ear canal so the surgeon cannot observe the healing and the patient cannot hear out of that ear. The use of the disclosed post-surgical implant device after completion of ear canal surgical operations or other treatment solves both those problems. The disclosed post-surgical implant device can utilize an open mesh or other suitable structures, which allows a practitioner to see the ear canal skin and ear drum, so that the condition of the ear canal skin and ear drum can be monitored after surgery or treatment and while it is healing. In addition, the patient can hear, wear a hearing aid and perform other tasks that would be impossible if Gelfoam® was used.

The disclosed post-surgical implant device can be implemented using the Propel® device and applicator, which can be modified to optimize its function for use in the ear canal. In one example embodiment, substances other than mometasone can be used, such as other suitable steroids, medications for auditory processing disorder (such as dexmethylphenidate), medications for Eustachian tube dysfunction, medications for Meniere's Disease, medications for Otitis Externa (such as ciprofloxacin), medications for Otitis Media, ototopical antibiotics or other suitable substances. In addition, the size and structure of the disclosed post-surgical implant device can be optimized to fit different sized ear canals (such as by increasing or decreasing the diameter or height), by shortening the applicator relative to the length of the Propel® device to make it easier to use in the shorter ear canal, then to dissolve sooner when needed or to have other suitable properties.

In one example embodiment, the following procedure can be used. First, the ear is prepared for surgery using conventional cleaning and disinfectants. After surgery or other treatment, the ear is rinsed with a sterilization compound, an antibiotic compound, an anesthetic compound, a lubricating compound, other suitable compounds or a combination of such compounds, and the disclosed post-surgical implant device is inserted into the ear canal using an applicator and is positioned in a location adjacent to the location of the surgery or other treatment. A subsequent rinse can then be applied, and the ear and disclosed post-surgical implant device is inspected to ensure that it has been properly located. The patient's hearing aid can then be fitted, and can be removed as needed for reapplication of an antibiotic compound, an anesthetic compound or other suitable medicines or compounds. A remote camera can be used for telehealth follow-up purposes, to eliminate the need for the patient to return to the doctor's office. The remote camera can include a wired or wireless connection to a computer, smart phone or other suitable processors, and can operate under algorithmic control to generate image data using white light, infra-red light, laser light at predetermined frequencies or other suitable illumination, to allow medical diagnosis to be remotely performed. The remote camera can also or alternatively be fitted by a practitioner prior to discharging the patient after surgery, to ensure that the camera will intrude too far into the ear canal.

To install the device, the implant and delivery system can be removed from a protective packaging using sterile procedures, and can then be inspected for any damage. If a brace is used, the brace should be attached to the distal end of the delivery system. The implant should normally be compressed and loaded into the tip of the delivery system prior to use. Next, the implant should be gently slid off its holder, after which the implant should be grasped between the fingers of both hands, to allow the implant to be gently compressed. The compressed implant can then be inserted into the brace as far as possible if it is attached to the distal tip of the delivery system, using gentle pressure applied by a fingertip or in other suitable manners. Afterward implantation, the brace can be carefully removed in a manner that does not to dislodge the implant. The brace can be replaced and the tip of the delivery system can be gently squeezed to hold the implant in place if the implant begins to withdraw from the tip when the brace is being removed. The implant should not remain in the undeployed state for more than two to three minutes prior to placement, but can be re-compressed and loaded into the delivery system tip one additional time if the first attempt is unsuccessful.

The top of the device can be curved during insertion, and can be smaller at the top/front end due to the a shape of a funnel at a tip of an applicator. The device can be formed from a flexible mesh-like stent that can be deployed into the ear canal that is the length of the ear canal or that covers the majority of the length of the ear canal or the operated or injured area. The stent can also be drug-eluding and/or bio-absorbable. The device can have a flexible structure with crossing, angled side structures and without a separate structure for a base or top, or other suitable configurations.

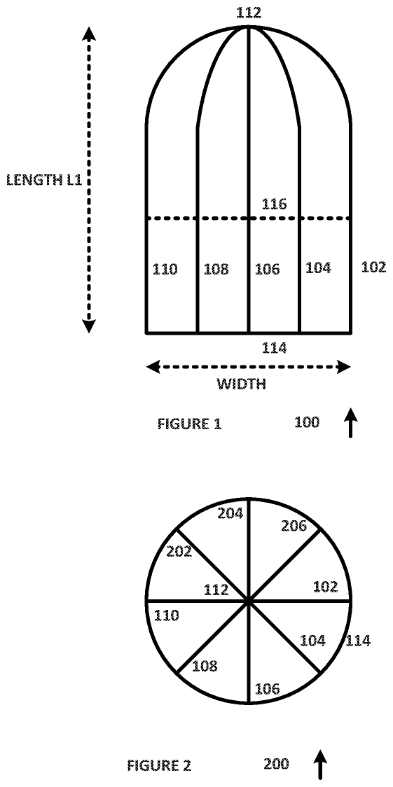

FIG. 1 is a side view of a surgical implant 100, in accordance with an example embodiment of the present disclosure. Surgical implant 100 includes sides 102 through 110, which are coupled to base 114 and which curve to meet at top 112 in an undeployed configuration, to facilitate installation into the ear canal or other locations. The specific dimensions of surgical implant 100 can be selected to conform to the specific application, but in one example embodiment, the length L1 of surgical implant 100 can be approximately 2.5 cm for most aural implant applications, but with a distribution of lengths between 2 to 3 cm depending on the specific dimensions of the patient's ear canal. Alternatively, the length L1 of the surgical implant can extend the length of the area operated on or the injured area of the ear canal, which can be less than 3 cm or less than 2 cm. Likewise, the width or diameter of surgical implant 100 can be approximately 0.7 cm for most aural implant applications, but with a distribution of lengths ranging from 0.5 to 1 cm depending on the specific dimensions of the patient's ear canal. In one example embodiment, the patient's ear canal or other suitable dimensions can be measured using a laser measurement device, an optical measurement device, a sonic measurement device or other suitable measurement devices to generate a 3D dimension map of the ear canal (or other suitable location), and the 3D dimension map can be used to print a customized implant, such as by using a polymer 3D printing process such as vat polymerization, material extrusion, powder bed fusion, material jetting or other suitable polymer 3D printing techniques.

While example dimensions have been provided for an ear canal, there are other suitable applications for surgical implant 100, including but not limited to arterial implants, digestive tract implants, wound implants, arthroscopic implants or other suitable applications where a dissolvable implant would be useful to provide a short period of time during which a scope, camera or other device can be used or implanted to allow the progressions of healing to be monitored, to allow addition medications to be delivered, to allow additional treatments to be applied (such as radiological, electrical or thermal) and for other suitable purposes.

Surgical implant 100 can also include additional structural supports 116, to improve the structural stability of surgical implant 100. While the structural support 116 is shown as being disposed circumferentially at the mid-point of the straight portion, other suitable configurations can also or alternatively be used, such as web supports, cross-supports, staggered supports, irregular supports and so forth.

The specific dimensions and materials of sides 102 through 110, base 114 and any other components of surgical implant 100 can be identical or can be varied as needed to provide suitable stability and access. In one example embodiment, the thickness of the sides 102 through 110, base 114 and any other components of surgical implant 100 can range from less than 1 mm to greater than 2 mm, such as where the thickness changes as a function of location. For example, the sides 102 through 110 can taper towards the top 112, to prevent the top 112 from forming a solid barrier. Likewise, the top 112 can be solid with one or more penetrations to facilitate viewing, aspiration or other functions. The materials that each component is made from can also be varied, such as to control a rate at which the component is disintegrated so that the structural configuration of implant 100 changes according to a controlled process. For example, it may be desirable for the top 112 to disintegrate before the base, and for the remainder of surgical implant 100 to disintegrate progressively, such as to prevent surgical implant 100 from collapsing in an uncontrolled and possibly temporarily painful configuration (e.g. with one or more projecting ends). Implant 100 can also be impregnated with medicine or other compounds that are released as the implant dissolves. For example, the implant 100 can be impregnated or coated with anti-inflammatory medicines. Implant 100 can be fabricated by injection molding, micromachining, compression molding, 3D material printing or in other suitable manners.

FIG. 2 is a bottom view of a surgical implant 200, in accordance with an example embodiment of the present disclosure. Surgical implant 200 is similar to surgical implant 100, and includes additional sides 202 through 206. In addition, base 114 is circular as shown, but other suitable shapes can be used for base 114, such as ellipses, irregular, square, rectangular and so forth.

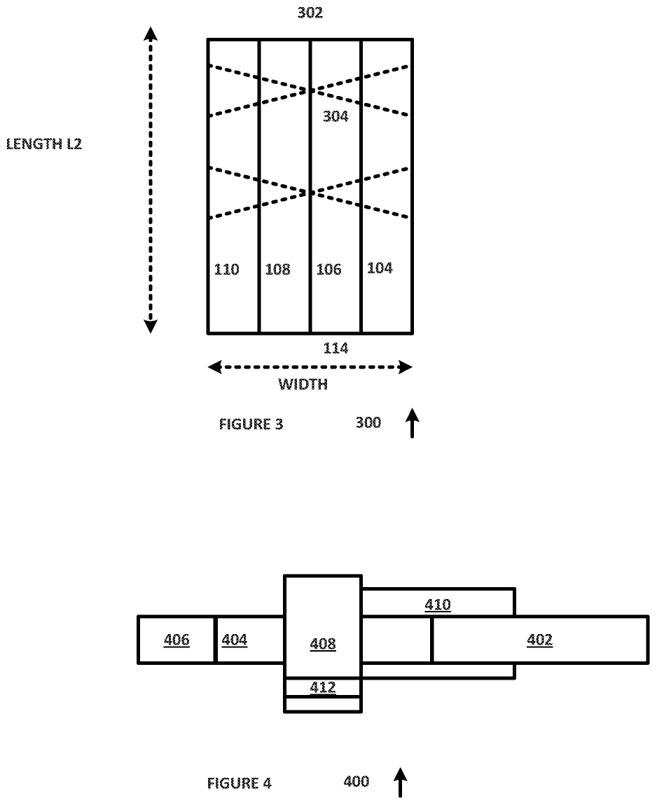

FIG. 3 is a side view of a deployed surgical implant 300, in accordance with an example embodiment of the present disclosure. Surgical implant 300 includes sides 102 through 110, which are coupled to base 114 and which are coupled at top 302. The specific dimensions of surgical implant 300 can be selected to conform to the specific application, but in one example embodiment, the length L2 of surgical implant 300 can be approximately 2.5 cm for most aural implant applications, but with a distribution of lengths between 2 to 3 cm depending on the specific dimensions of the patient's ear canal. As such, the length is substantially less for aural applications than might be appropriate for other applications, which could seriously injure the eardrum if those were used unless modified in accordance with the teachings of the present disclosure. Likewise, the width or diameter of surgical implant 300 can be approximately 0.7 cm for most aural implant applications, but with a distribution of lengths ranging from 0.5 to 1 cm depending on the specific dimensions of the patient's ear canal. In one example embodiment, the patient's ear canal or other suitable dimensions can be measured using a laser measurement device, an optical measurement device, a sonic measurement device or other suitable measurement devices to generate a 3D dimension map of the ear canal (or other suitable location), and the 3D dimension map can be used to print a customized implant, such as by using a polymer 3D printing process such as vat polymerization, material extrusion, powder bed fusion, material jetting or other suitable polymer 3D printing techniques.

While example dimensions have been provided for an ear canal, there are other suitable applications for surgical implant 300, including but not limited to arterial implants, digestive tract implants, wound implants, arthroscopic implants or other suitable applications where a dissolvable implant would be useful to provide a short period of time during which a scope, camera or other device can be used or implanted to allow the progressions of healing to be monitored, to allow addition medications to be delivered, to allow additional treatments to be applied (such as radiological, electrical or thermal) and for other suitable purposes.

Surgical implant 300 can also include additional structural supports 304, to improve the structural stability of surgical implant 300. While the structural supports 304 are shown as being disposed as cross-supports, other suitable configurations can also or alternatively be used, such as web supports, circumferential supports at the mid-point of the straight portion, staggered supports, irregular supports and so forth.

The specific dimensions and materials of sides 102 through 110, base 114 and any other components of surgical implant 300 can be identical or can be varied as needed to provide suitable stability and access. In one example embodiment, the thickness of the sides 102 through 110, base 114 and any other components of surgical implant 300 can range from less than 1 mm to greater than 2 mm, such as where the thickness changes as a function of location. The materials that each component is made from can also be varied, such as to control a rate at which the component is disintegrated so that the structural configuration of implant 300 changes according to a controlled process. For example, it may be desirable for the top 112 to disintegrate before the base, and for the remainder of surgical implant 300 to disintegrate progressively, such as to prevent surgical implant 300 from collapsing in an uncontrolled and possibly temporarily painful configuration (e.g. with one or more projecting ends). Implant 300 can also be impregnated with medicine or other compounds that are released as the implant dissolves. Implant 300 can be fabricated by injection molding, micromachining, compression molding, 3D material printing or in other suitable manners.

FIG. 4 is a side view of a delivery system 400, in accordance with an example embodiment of the present disclosure. Delivery system 400 includes plunger 402, support 404, implant 406, brace 408 and housing 410, each of which can be made from a polymer, an elastomeric, a rubber, a composite, a combination of these materials or other suitable materials.

Plunger 402 is coupled to support 404, which holds implant 406. Support 404 and implant 406 are inserted through brace 408, until housing 410 abuts brace 408. The combined length of implant 406 and support 404 which extends past brace 408 should typically not exceed 30 millimeters, as that is the normal length of an ear canal. Exceeding that length can cause injury to a patient. Brace 408 can include an endoscope port 412 to facilitate endoscopic viewing of the application of implant 406. Plunger 402 and support 404 can include a release mechanism, such as a channel for a fluid that causes implant 406 to be release, or other suitable mechanisms. Brace 408 is configured to be placed against a patient's ear, and can include a port 412 to allow an endoscope to be inserted for viewing of the deployment of implant 406.

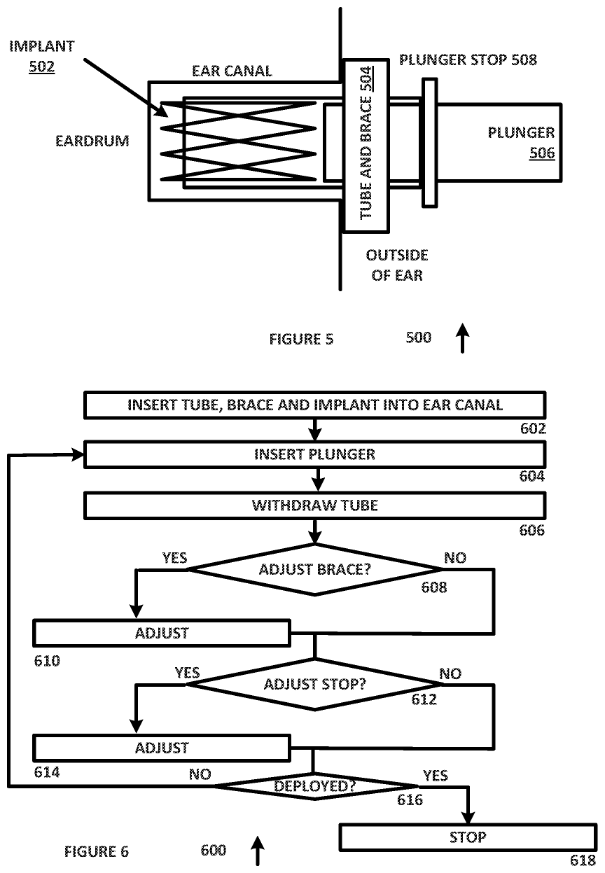

FIG. 5 is a diagram of a surgical implant delivery system 500 in an ear canal with a surgical implant, in accordance with an example embodiment of the present disclosure. System 500 includes implant 502, tube and brace 504, plunger 506 and plunger stop 508, each of which can be fabricated from a polymer, an elastomeric, a rubber, a composite, a combination of these materials or other suitable materials, as discussed further herein.

Implant 502 can be formed from a plurality of supports, where each support is coupled at a first end to one adjacent support and at a second end to a second adjacent support, so as to form an accordion-type spring that can expand when not compressed. A suitable joint between each support can allow the supports to rotate relative to the adjacent support, and the supports can form a tube that can be compressed. The support can be impregnated with medicine or other compounds that are released as the implant dissolves. For example, the supports of implant 502 can be impregnated or coated with anti-inflammatory medicines. Implant 502 can be fabricated by injection molding, micromachining, compression molding, 3D material printing or in other suitable manners.

Tube and brace 504 are configured to be inserted into an ear canal as shown, where the brace is movable along the length of the tube, and where implant 502 is inserted into the tube by compressing implant 502 and inserting it into the tube. The brace is positioned to prevent the tube from being inserted into the ear canal to a point where the patient's ear drum could be damaged.

Plunger 506 is configured to be inserted into the tube of tube and brace 504, to allow implant 502 to be pushed out of the tube. Plunger stop 508 is a movable stop on plunger 506 that prevents plunger 506 from causing implant 502 to be pushed into the patient's eardrum.

In operation, a practitioner deploys implant 502 by inserting tube and brace 504 into a patient's ear canal and then by pushing implant 502 out of the tube using plunger 506. Plunger stop 508 and the brace of tube and brace 504 are used to control the extent to which the tube, plunger 506 and implant 502 can be inserted, so as to aid in preventing the inadvertent injury of the patient's ear drum.

FIG. 6 is a diagram of an method 600 for utilizing a surgical implant delivery system in an ear canal with a surgical implant, in accordance with an example embodiment of the present disclosure. Method 600 begins at 602 where a tube, brace and implant are adjusted for insertion into an ear canal and are then inserted. By adjusting the position of the brace and the implant, the patient's ear drum can be protected from inadvertent injury. The method then proceeds to 604, where a plunger is inserted to cause the implant to be deployed from the tube. The method then proceeds to 606.

At 606, the tube is withdrawn from the ear canal. In one example embodiment, the tube can be withdrawn in a coordinated manner with the insertion of the plunger, so as to deploy the insert into the patient's ear canal. The method then proceeds to 608.

At 608, it is determined whether a brace should be adjusted. In one example embodiment, the brace can be adjusted after the tube has been withdrawn a partial distance and the implant has been deployed a partial distance, so as to prevent the tube and implant from being inserted into the patient's ear canal and damaging the patient's ear drum. If it is determined that the brace should be adjusted, the method then proceeds to 610, where the brace is adjusted, otherwise the method proceeds to 612.

At 612, it is determined whether a plunger stop should be adjusted. In one example embodiment, the plunger stop can be adjusted after the tube has been withdrawn a partial distance and the implant has been deployed a partial distance, so as to prevent the tube and implant from being inserted into the patient's ear canal and damaging the patient's ear drum. If it is determined that the plunger stop should be adjusted, the method then proceeds to 614, where the plunger stop is adjusted, otherwise the method proceeds to 616.

At 616, it is determined whether the implant is deployed. If it is determined that the implant has been deployed, the method then proceeds to 618 and terminates, otherwise the method returns to 604.

As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

As used herein, “hardware” can include a combination of discrete components, an integrated circuit, an application-specific integrated circuit, a field programmable gate array, or other suitable hardware. As used herein, “software” can include one or more objects, agents, threads, lines of code, subroutines, separate software applications, two or more lines of code or other suitable software structures operating in two or more software applications, on one or more processors (where a processor includes one or more microcomputers or other suitable data processing units, memory devices, input-output devices, displays, data input devices such as a keyboard or a mouse, peripherals such as printers and speakers, associated drivers, control cards, power sources, network devices, docking station devices, or other suitable devices operating under control of software systems in conjunction with the processor or other devices), or other suitable software structures. In one exemplary embodiment, software can include one or more lines of code or other suitable software structures operating in a general purpose software application, such as an operating system, and one or more lines of code or other suitable software structures operating in a specific purpose software application. As used herein, the term “couple” and its cognate terms, such as “couples” and “coupled,” can include a physical connection (such as a copper conductor), a virtual connection (such as through randomly assigned memory locations of a data memory device), a logical connection (such as through logical gates of a semiconducting device), other suitable connections, or a suitable combination of such connections. The term “data” can refer to a suitable structure for using, conveying or storing data, such as a data field, a data buffer, a data message having the data value and sender/receiver address data, a control message having the data value and one or more operators that cause the receiving system or component to perform a function using the data, or other suitable hardware or software components for the electronic processing of data.

In general, a software system is a system that operates on a processor to perform predetermined functions in response to predetermined data fields. A software system is typically created as an algorithmic source code by a human programmer, and the source code algorithm is then compiled into a machine language algorithm with the source code algorithm functions, and linked to the specific input/output devices, dynamic link libraries and other specific hardware and software components of a processor, which converts the processor from a general purpose processor into a specific purpose processor. This well-known process for implementing an algorithm using a processor should require no explanation for one of even rudimentary skill in the art. For example, a system can be defined by the function it performs and the data fields that it performs the function on. As used herein, a NAME system, where NAME is typically the name of the general function that is performed by the system, refers to a software system that is configured to operate on a processor and to perform the disclosed function on the disclosed data fields. A system can receive one or more data inputs, such as data fields, user-entered data, control data in response to a user prompt or other suitable data, and can determine an action to take based on an algorithm, such as to proceed to a next algorithmic step if data is received, to repeat a prompt if data is not received, to perform a mathematical operation on two data fields, to sort or display data fields or to perform other suitable well-known algorithmic functions. Unless a specific algorithm is disclosed, then any suitable algorithm that would be known to one of skill in the art for performing the function using the associated data fields is contemplated as falling within the scope of the disclosure. For example, a message system that generates a message that includes a sender address field, a recipient address field and a message field would encompass software operating on a processor that can obtain the sender address field, recipient address field and message field from a suitable system or device of the processor, such as a buffer device or buffer system, can assemble the sender address field, recipient address field and message field into a suitable electronic message format (such as an electronic mail message, a TCP/IP message or any other suitable message format that has a sender address field, a recipient address field and message field), and can transmit the electronic message using electronic messaging systems and devices of the processor over a communications medium, such as a network. One of ordinary skill in the art would be able to provide the specific coding for a specific application based on the foregoing disclosure, which is intended to set forth exemplary embodiments of the present disclosure, and not to provide a tutorial for someone having less than ordinary skill in the art, such as someone who is unfamiliar with programming or processors in a suitable programming language. A specific algorithm for performing a function can be provided in a flow chart form or in other suitable formats, where the data fields and associated functions can be set forth in an exemplary order of operations, where the order can be rearranged as suitable and is not intended to be limiting unless explicitly stated to be limiting.

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

What is claimed is:

1. A device for post-operative ear canal treatment, comprising:
a plurality of sides, each having a length of less than 5 millimeters, the plurality of sides coupled to an adjacent side at a first end and to another adjacent side at a second end, the plurality of sides forming a compressible tube; and
a delivery system configured to controllably install the device in the ear canal with a restriction on the extent to which the delivery system can be inserted into the ear canal, so as to prevent injury to an ear drum.

2. The device of claim 1 wherein the delivery system comprises a tube.

3. The device of claim 1 wherein the delivery system comprises a plunger.

4. The device of claim 1 wherein the delivery system comprises:
a tube; and
a movable brace disposed on the tube.

5. The device of claim 1 wherein the delivery system comprises:
a plunger; and
a movable stop disposed on the plunger.

6. The device of claim 1 wherein the delivery system comprises:
a tube;
a movable brace disposed on the tube;
a plunger; and
a movable stop disposed on the plunger.

7. The device of claim 1 wherein the delivery system comprises:
a tube;
a movable brace disposed on the tube;
the device disposed in the tube;
a plunger; and
a movable stop disposed on the plunger.

8. The device of claim 1 wherein the plurality of sides are each formed from a material that dissolves over time when exposed to an ear canal and which releases a predetermined ear medication as it dissolves.

9. A method for post-operative ear canal treatment, comprising:
inserting a device having a length of less than 5 millimeters into a tube;
inserting the tube into an ear canal without contacting an ear drum;
inserting a plunger into the tube; and
deploying the device in the ear canal by pushing the device out of the tube with the plunger while withdrawing the tube from the ear canal.

10. The method of claim 9 comprising adjusting a brace on the tube prior to inserting the tube into the ear canal.

11. The method of claim 9 comprising adjusting a brace on the tube after partially removing the tube from the ear canal.

12. The method of claim 9 comprising adjusting a stop on the plunger prior to inserting the plunger into the tube.

13. The method of claim 9 comprising adjusting a stop on the plunger after partially removing the tube from the ear canal.

14. The method of claim 9 comprising:
adjusting a brace on the tube prior to inserting the tube into the ear canal; and
adjusting the brace on the tube after partially removing the tube from the ear canal.

15. The method of claim 9 comprising:
adjusting a stop on the plunger prior to inserting the plunger into the tube; and
adjusting the stop on the plunger after partially removing the tube from the ear canal.

16. The method of claim 9 comprising:
adjusting a brace on the tube prior to inserting the tube into the ear canal;
adjusting the brace on the tube after partially removing the tube from the ear canal;
adjusting a stop on the plunger prior to inserting the plunger into the tube; and
adjusting the stop on the plunger after partially removing the tube from the ear canal.

17. The method of claim 9 comprising removing the tube from the ear canal after the device has been deployed.

18. Performing a method with a device for post-operative ear canal treatment that includes a plurality of sides, each having a length of less than 5 millimeters, the plurality of sides coupled to an adjacent side at a first end and to another adjacent side at a second end, the plurality of side forming a compressible tubular, the plurality of sides each formed from a material that dissolves over time when exposed to an ear canal and which releases a predetermined ear medication as it dissolves and a delivery system configured to controllably install the device in the ear canal with a restriction on the extent to which the delivery system can be inserted into the ear canal, so as to prevent injury to an ear drum, wherein the delivery system comprises a tube, a movable brace disposed on the tube, the device disposed in the tube, a plunger and a movable stop disposed on the plunger, the method comprising:
inserting the device into the tube;
inserting the tube into the ear canal without contacting the ear drum;
inserting the plunger into the tube;
deploying the device in the ear canal by pushing the device out of the tube with the plunger while withdrawing the tube from the ear canal;
adjusting the brace on the tube prior to inserting the tube into the ear canal;
adjusting the brace on the tube after partially removing the tube from the ear canal;
adjusting the stop on the plunger prior to inserting the plunger into the tube;
adjusting the stop on the plunger after partially removing the tube from the ear canal; and
removing the tube from the ear canal after the device has been deployed.
